(19) United States
(12) Patent Application Publication
(10) Pub. No.: US 2024/0269418 A1
(43) Pub. Date: Aug. 15, 2024

(54) SAMPLE GAS FILTER FOR A THERAPEUTIC GAS DELIVERY DEVICE

(71) Applicant: Mallinckrodt Pharmaceuticals Ireland Limited, Dublin (IE)

(72) Inventors: Eoin Cannon, Perrystown (IE); Michael McAtamney, Navan (IE)

(21) Appl. No.: 18/441,216

(22) Filed: Feb. 14, 2024

Related U.S. Application Data
(60) Provisional application No. 63/445,418, filed on Feb. 14, 2023.

(51) Int. Cl. A61M 16/08; A61M 16/10

(52) U.S. Cl. CPC A61M 16/0808; A61M 16/085; A61M 16/105; A61M 2202/0275; A61M 2205/3327; A61M 2205/7527

(57) ABSTRACT

Disclosed herein is a sample gas filter for a therapeutic gas delivery device. The sample gas filter can include a housing having a sample gas inlet, which can receive a sample gas from a sample line connected to an inspiratory line of the therapeutic gas delivery device, and a sample gas outlet. The housing can include a first chamber having a first filter membrane and a first reservoir between the sample gas inlet and the first filter membrane. The housing can include a second chamber having a second filter membrane and a second reservoir between the first filter membrane and the sample gas outlet. The first reservoir and the second reservoir can be oriented axially so that the sample gas filter is operable in any axial orientation. The sample gas filter can remove water vapor from the sample gas and collect water in the first and/or second reservoir.

### CLAIM OF PRIORITY

This application claims the benefit of U.S. Provisional Application No. 63/445,418, filed on Feb. 14, 2023, the entire contents of which are herein incorporated by reference in its entirety.

### FIELD

The present disclosure is directed to a patient gas sample line and filter and methods of use thereof. More specifically, the present disclosure is directed to a patient gas sample line and filter that includes a multi-stage filtration system to filter water vapor from sample gas in a therapeutic gas delivery system.

### BACKGROUND

Therapeutic gas can be delivered to patients through inspiratory breathing gas flowing from a breathing circuit affiliated with a ventilator. For example, the therapeutic gas can be injected into inspiratory breathing gas flowing in the breathing circuit and, subsequently, delivered to the airways of the patient. One such therapeutic gas is nitric oxide, which can produce vasodilatory effects on a patient.

While administering therapeutic gas, a sampling system can monitor a portion of the inspiratory breathing gas to confirm that the therapeutic gas is being delivered at a desired dose in the inspiratory breathing gas flow. For example, a patient gas sample line and filter can be used to provide sample gas (e.g., a portion of the inspiratory breathing gas flow) to a gas sensor module, which monitors the concentrations of the therapeutic gas being delivered to the patient. In some cases, the breathing circuit, which delivers the therapeutic gas to the airways of the patient, can be humidified. Traditionally, the patient gas sample line and filter involved a complex design to separate the liquid from the sample gas. However, these designs can be difficult to manufacture and can require the patient gas sample line and filter to be in a specific orientation during use. Moreover, these designs can allow wicking between various stages of filtration, which can cause premature occlusion.

Therefore, there is a need for a patient gas sample line and filter that is more convenient to manufacture, is more convenient to use, and is long-lasting.

### SUMMARY

Aspects of the present disclosure include a sample gas filter for a therapeutic gas delivery device. The sample gas filter can include a housing, a first chamber, and a second chamber. The housing can have a sample gas inlet, which can receive a sample gas from a sample line connected to an inspiratory line of the therapeutic gas delivery device, and a sample gas outlet. The first chamber can be within the housing and can include a first filter membrane and a first reservoir. The first reservoir can be located between the sample gas inlet and the first filter membrane. The second chamber can be within the housing and can include a second filter membrane and a second reservoir. The second reservoir can be between the first filter membrane and the sample gas outlet. The first reservoir and the second reservoir can be oriented axially so that the sample gas filter can be used in any axial orientation. The sample gas filter can remove water vapor from the sample gas line and can collect water in the first reservoir and/or second reservoir.

In certain instances, the first chamber can include a baffle plate and a fiber membrane. The baffle plate can be operable to support the first filter membrane on a first side of the first filter membrane and the fiber membrane can be operable to support the first filter membrane on a second side of the first filter membrane.

In certain instances, the second chamber can include a press-fit baffle and a labyrinth support. The press-fit baffle can support the second filter membrane on a first side of the second filter membrane. The labyrinth support can be located on a wall of the housing having the sample gas outlet. The labyrinth support can support the second filter membrane on a second side of the second filter membrane.

In certain instances, the housing and each of the chambers can have a substantially circular cross-section. In certain instances, the first filter membrane and the second filter membrane can be substantially circular.

In certain instances, the first filter membrane can have a larger diameter than the second filter membrane. In certain instances, the first reservoir can have a larger volume than the second reservoir. In certain instances, the first reservoir and the second reservoir can be large enough to accommodate water for twelve hours of continuous use.

In certain instances, the first filter membrane can be a glass fiber filter membrane. In certain instances, the second filter membrane can be a 0.22 μm PTFE membrane. In certain instances, the fiber membrane can be made of course sintered porous plastic materials (in one example, Vyon® fiber membrane). In certain instances, the first filter membrane and the second filter membrane can be separated to prevent wicking between the membranes.

Aspects of the present disclosure include a sample gas filter for a therapeutic gas delivery device. The sample gas filter can include a housing, a first chamber, and a second chamber. The housing can have a sample gas inlet, which can receive a sample gas from a sample line connected to an inspiratory line of the therapeutic gas delivery device, and a sample gas outlet. The first chamber can be within the housing and can include a first filter membrane, a baffle plate, a fiber membrane, and a first reservoir. The first filter membrane can have a first side and a second side. The baffle plate can support the first filter membrane on the first side and the fiber membrane can support the first filter membrane on the second side. The first reservoir can be between the sample gas inlet and the baffle plate. The second chamber can be within the housing and can include a second filter membrane, a press-fit baffle, a labyrinth support, and a second reservoir. The second filter membrane can have a first side and a second side. The press-fit baffle can support the second filter membrane on the first side. The labyrinth support can be on a wall of the housing that has the sample gas outlet and the labyrinth support can support the second filter membrane on the second side. The second reservoir can be between the fiber membrane of the first chamber and the press-fit baffle.

In certain instances, the sample gas filter can remove water vapor from the sample gas and collect water in the first reservoir and/or the second reservoir.

In certain instances, the housing and each of the chambers can have a substantially circular cross-section.

In certain instances, the first reservoir and the second reservoir can be oriented axially so that the sample gas filter can be used in any axial orientation.

In certain instances, the first filter membrane and the second filter membrane can be substantially circular.

In certain instances, the first filter membrane can have a larger diameter than the second filter membrane.

In certain instances, the first reservoir can have a larger volume than the second reservoir.

In certain instances, the first reservoir and the second reservoir can be large enough to accommodate water for twelve hours of continuous use.

In certain instances, the first filter membrane can be a glass fiber filter membrane. In certain instances, the second filter membrane can be a 0.22 μm PTFE filter membrane. In certain instances, the fiber membrane can be made of sintered porous plastic materials (in one example, a course Vyon® fiber membrane). In certain instances, the first filter membrane and the second filter membrane can be separated to prevent wicking between the membranes.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIGS. 1A-1B illustrate a therapeutic gas delivery system with a sample gas filter assembly.

FIGS. 2A-2F illustrate one instance of a sample gas filter assembly. FIG. 2A is a perspective view, FIG. 2B is a right-side view, FIG. 2C is a top view, FIG. 2D is a front-side view, and FIG. 2E is a cross-sectional view of the sample gas filter assembly. FIG. 2F is a zoomed in cross-sectional view of the sample gas filter.

FIG. 3A-3D illustrate one instance of a sample gas filter in perspective, cross-sectional views.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented.

The term “coupled” as used herein is defined as connected, whether directly or indirectly through intervening components, and is not necessarily limited to physical connections. The connection can be such that the objects are permanently connected or releasably connected.

The term “substantially” is defined to be essentially conforming to the particular dimension, shape or other word that substantially modifies, such that the component need not be exact.

The terms “comprising,” “including” and “having” are used interchangeably in this disclosure. The terms “comprising,” “including” and “having” mean to include, but not necessarily be limited to the things so described.

The terms “filter” and “filtration” are used herein in their broadest sense to encompass any and all of various types and degrees of removal or separation of liquid from gas, and may also include removal of other non-liquid particulates if present in some cases.

The present disclosure relates to a patient gas sample line and filter (e.g., sample gas filter assembly) and methods of use thereof. The filter includes a multi-stage filtration system with liquid reservoir, which filters liquid from sample gas (e.g., a portion of a mixture of the breathing gas and therapeutic gas) containing the liquid. The liquid component may be any removable liquid, such as, for example, humidity, water vapor, moisture from humidified air, other liquids in a vapor state, nebulized liquids, nebulized medical solutions and suspensions, etc.

The sample gas filter assembly can be used with a therapeutic gas delivery system that delivers a therapeutic gas (e.g., nitric oxide) to the airways of a patient. The therapeutic gas is delivered to the patient by dosing into the breathing circuit, usually in line with a mechanical ventilator. A subsystem of the therapeutic gas delivery system contains a gas sensor module with one or more gas sensors, which monitors the concentrations of the therapeutic gas and/or other gasses delivered to the patient. The gas sensor module is connected to the same patient breathing circuit as the therapeutic gas delivery system.

Oftentimes, the breathing circuit is humidified; accordingly, the gas sampled by the gas sensor module has a high percentage of water vapor. The present disclosure filters the water vapor from the sample gas and to collects it in a reservoir, while allowing the remainder of the sample gas to proceed to the one or more gas sensors within the gas sensor module. The sample gas filter assembly disclosed herein allows the device to operate at elevated humidity (e.g., when active humidification is used in the breathing circuit) for extended durations (e.g., twelve or more hours).

The sample gas filter assembly may be more convenient to use than a traditional patient gas sample line and filter. For example, the sample gas filter assembly may be easier to manufacture (e.g., it may involve only one or two manufacturing steps). Additionally, the sample gas filter assembly may be used in any axial orientation.

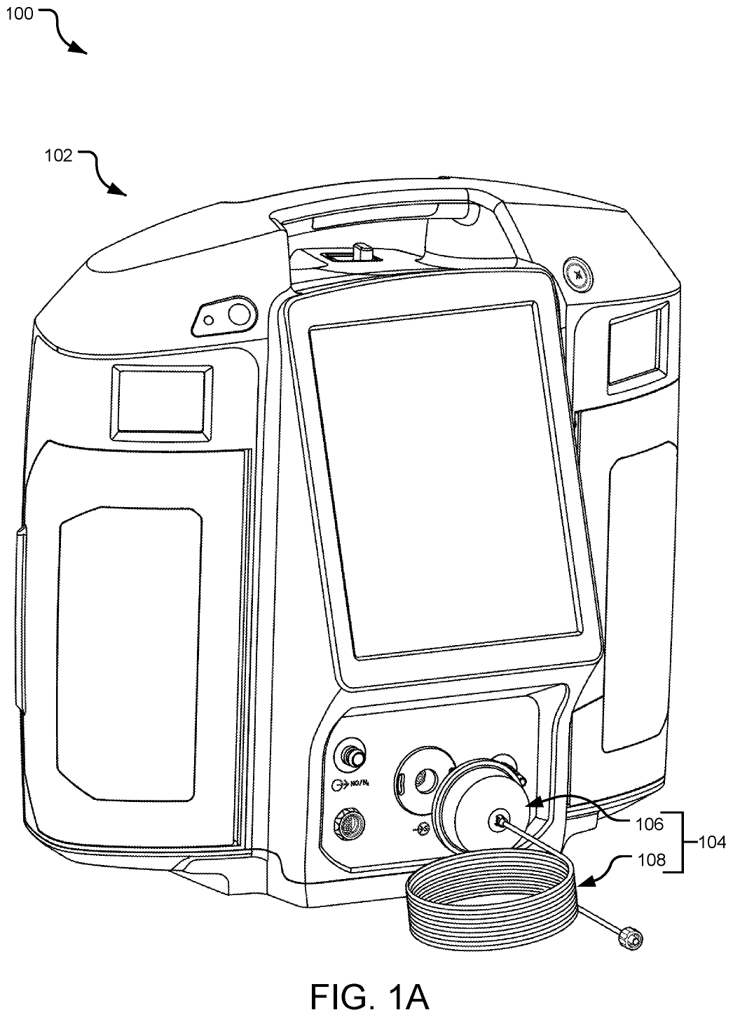

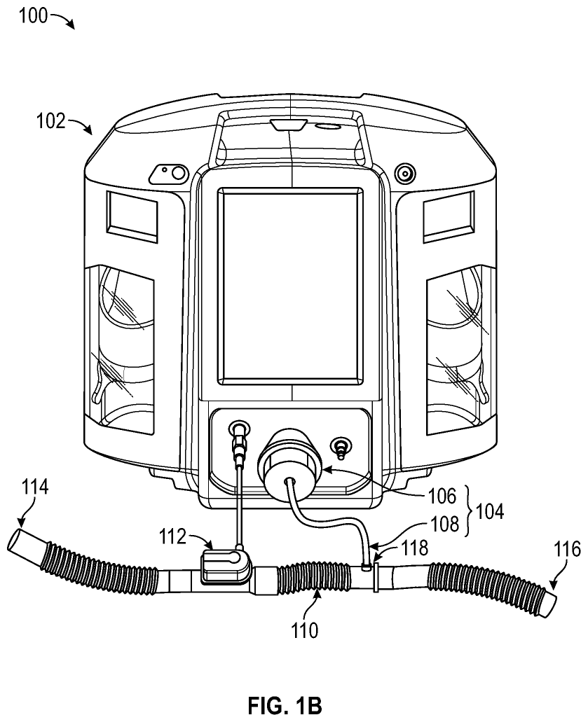

FIGS. 1A-1B illustrate one instance of a therapeutic gas delivery system 100 (e.g., a nitric oxide delivery system). The therapeutic gas delivery system 100 can include a therapeutic gas delivery device 102 and a sample gas filter assembly 104 (e.g., a patient gas sample line). The sample gas filter assembly 104 can include a sample gas filter 106 and a sample line 108. The sample gas filter assembly 104 is disconnected from the therapeutic gas delivery device 102 in FIG. 1A, while the sample gas filter assembly 104 is connected to the therapeutic gas delivery device in FIG. 1B.

The therapeutic gas delivery device 102 can deliver therapeutic gas (e.g., nitric oxide) to the airways of a patient. In one instance, a gas subsystem (not shown in the figures) of the therapeutic gas delivery device 102 can be in fluid communication with an inspiratory line 110 (e.g., breathing circuit tubing), thereby establishing a fluid flow path between the gas subsystem and the inspiratory line 110. For example, as illustrated in FIG. 1B, the gas subsystem can be in fluid communication with a gas injector module 112 and the gas injector module 112 can be in fluid communication with the inspiratory line 110. The inspiratory line 110 can include an upstream end 114 and a downstream end 116, which is opposite the upstream end 114. The upstream end 114 can be in fluid communication with, for example, a ventilator (not shown in the figures). The downstream end 116 can be in fluid communication with, for example, a patient (not shown in the figures). Thus, gas can flow from the gas subsystem of the therapeutic gas delivery device 102, into the inspiratory line 110, and into the airways of the patient.

The therapeutic gas delivery device 102 can also include a gas sensor module (not shown in the figures). The gas sensor module can include one or more gas sensors (e.g., nitric oxide sensor, nitrogen dioxide sensor, oxygen sensor) that can analyze sample gas (e.g., a portion of the gas that is being delivered to the patient). The sample gas can include, for example, breathing gas and therapeutic gas (e.g., nitric oxide) being delivered to the airways of the patient. In one instance, the sample gas filter assembly 104 can be in fluid communication with the inspiratory line 110 and the gas sensor module, thereby establishing a fluid flow path between the inspiratory line 110 and the gas sensor module of the therapeutic gas delivery device 102. For example, a sample tee 118 can be in fluid communication with the inspiratory line 110 and the sample tee 118 can be in fluid communication with the inspiratory line 110. The sample tee 118 can be located downstream from the gas injector module 112. Sample gas can flow from the inspiratory line 110, through the sample gas filter assembly 104, and to the gas sensor module of the therapeutic gas delivery device 102.

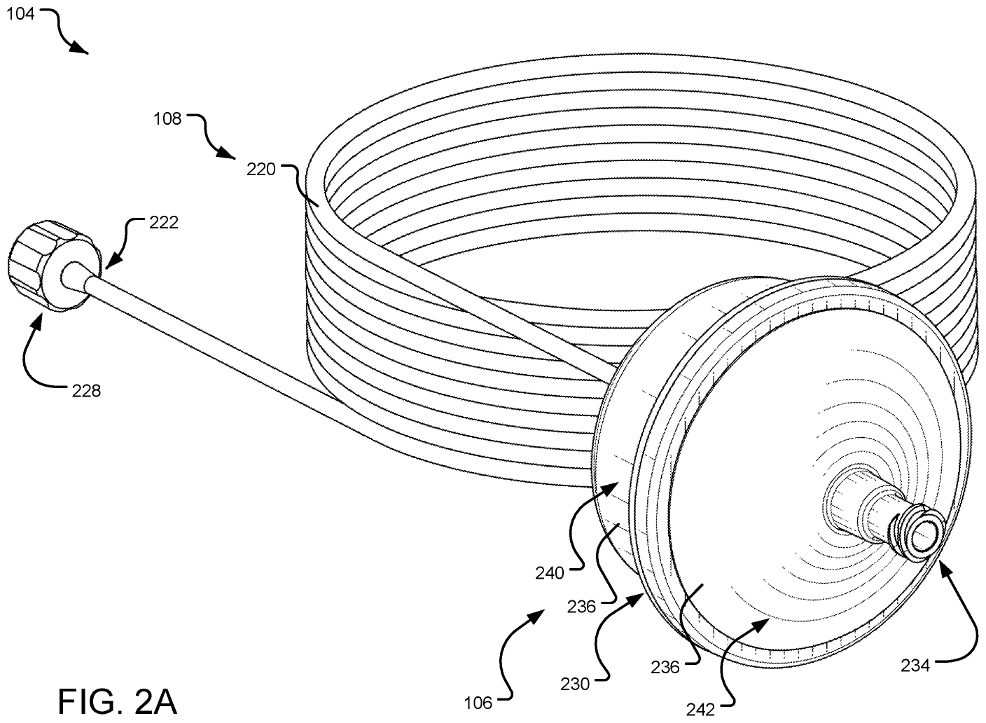

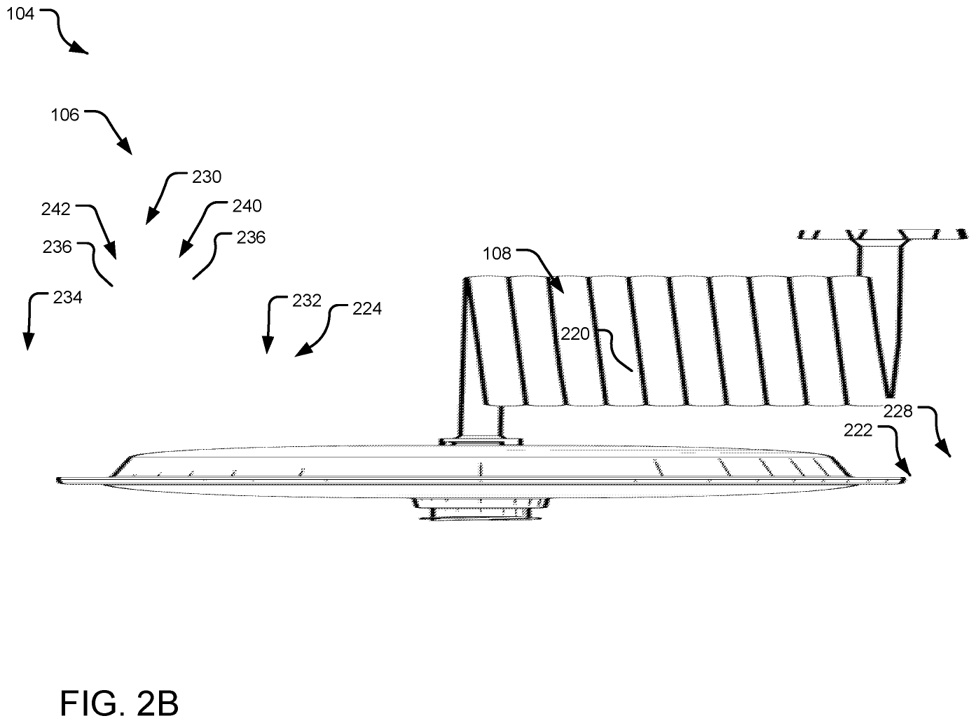

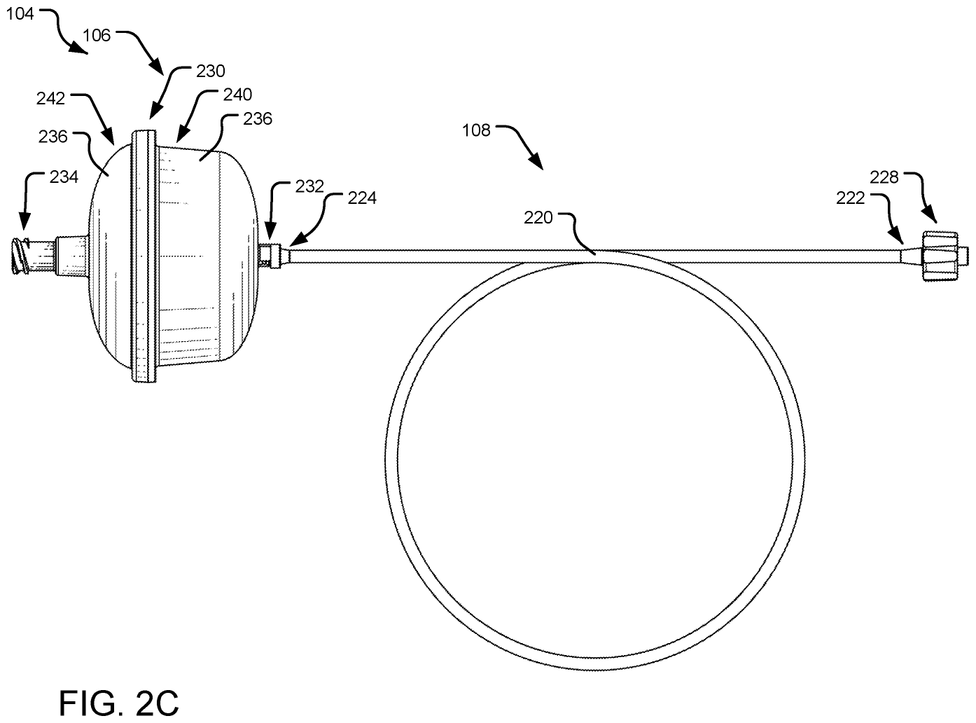

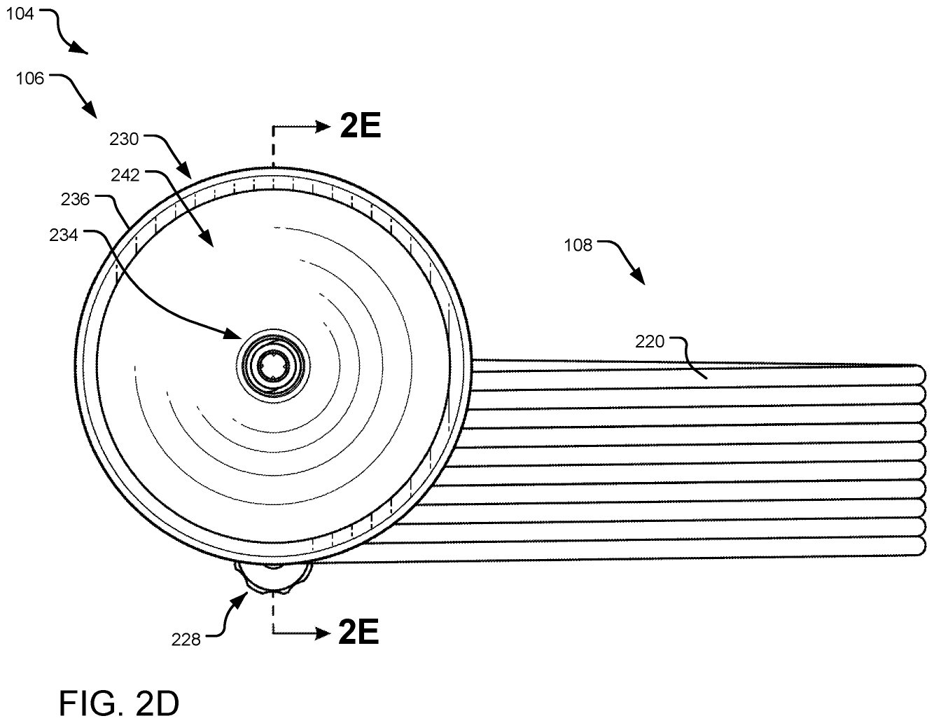

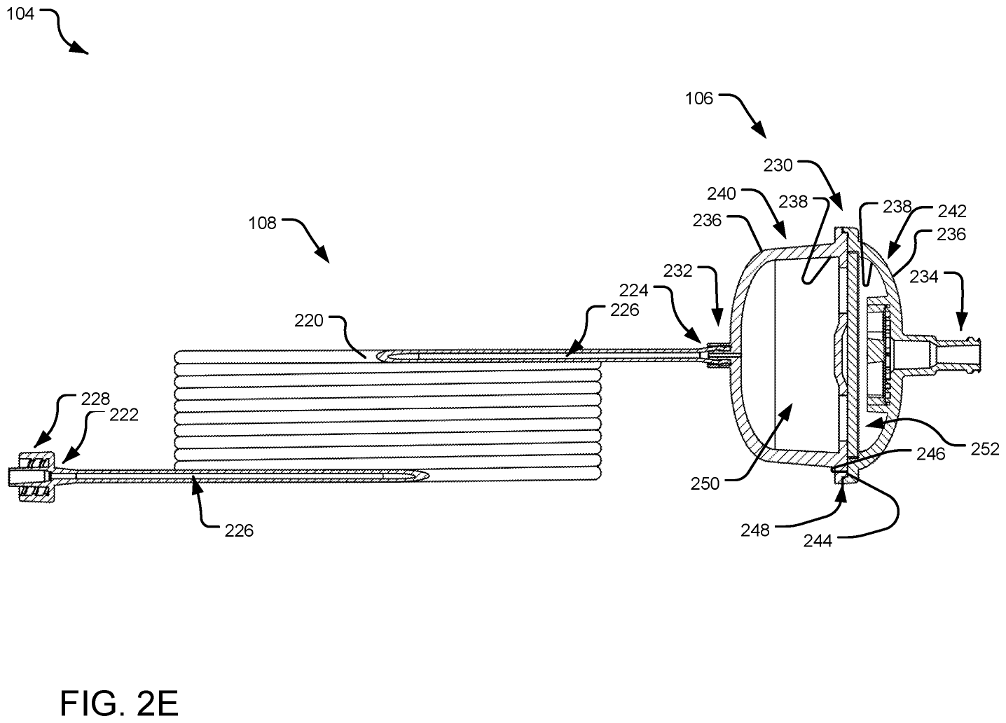

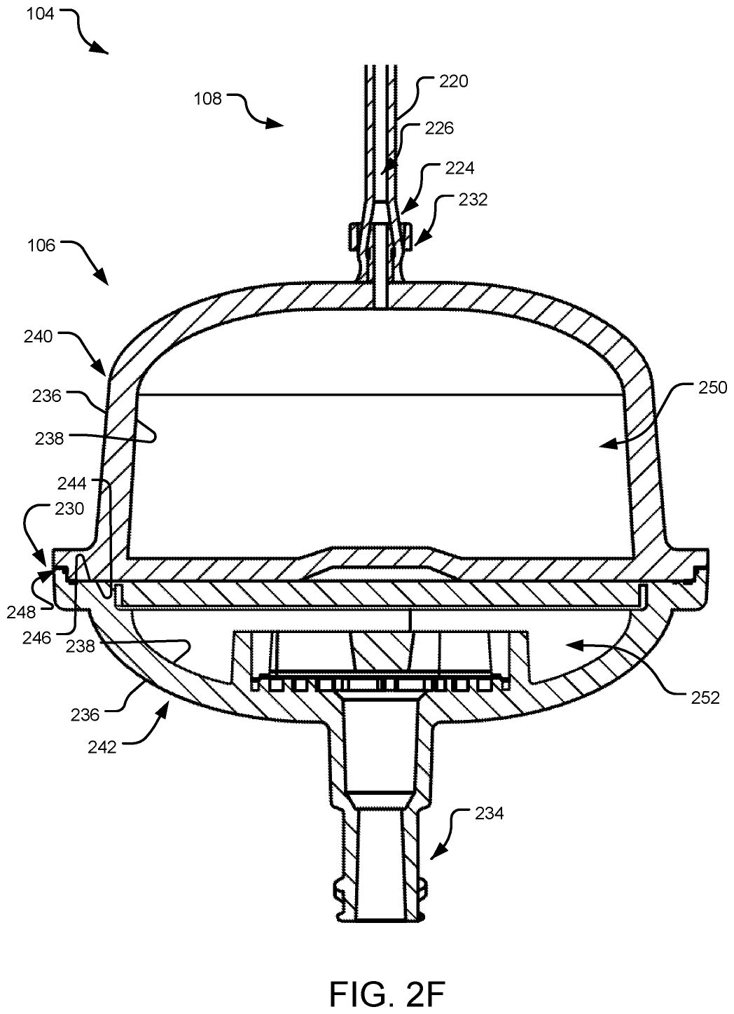

FIGS. 2A-2F illustrate one instance of a sample gas filter assembly 104. The sample gas filter assembly 104, as illustrated, for example, in the perspective view in FIG. 2A, can include a sample gas filter 106 and can also include a sample line 108. As discussed previously, the sample gas filter assembly 104 can be used in a therapeutic gas delivery device (not shown in FIGS. 2A-2F). For example, the sample gas filter assembly 104 can be used to establish fluid communication (e.g., establish a fluid flow path) between an inspiratory line (e.g., breathing circuit tubing) and a gas sensor module of the therapeutic gas delivery device.

Beginning with the sample line 108 of the sample gas filter assembly 104, the sample line 108 (e.g., a length of tubing) includes an elongated body 220 that has an inlet end 222 and an outlet end 224 opposite the inlet end 222, as illustrated, for example, in the side view of FIG. 2B. The elongated body 220 defines a central lumen 226, as illustrated, for example, in the cross-sectional views in FIGS. 2E-2F (which were taken along the cross-sectional lines illustrated in FIG. 2D). The central lumen 226 extends along the longitudinal axis of the elongated body 220 from the inlet end 222 to the outlet end 224. In other words, the inlet end 222 is in fluid communication with the outlet end 224, thereby establishing a fluid flow path through the central lumen 226 of the elongated body 220 of the sample line 108. The central lumen 226 can convey sample gas through the elongated body 220 (e.g., from the inlet end 222 to the outlet end 224).

The inlet end 222 of the sample line 108 can receive sample gas into the sample line 108. In one example, the inlet end 222 can receive sample gas from an inspiratory line (not shown in FIGS. 2A-2F) of a therapeutic gas delivery device (not shown in FIGS. 2A-2F). The inlet end 222 of the sample line 108 can include a connector 228 such as, for example, a luer fitting. The connector 228 can be configured to removably couple the sample line 108 to an inspiratory line (e.g., via a sample tee) to establish fluid communication (e.g., a fluid flow path) between the inspiratory line and the sample gas filter assembly 104.

The outlet end 224 of the sample line 108, as illustrated, for example, in the side view of the sample gas filter assembly 104 in FIG. 2B, can discharge sample gas from the sample line 108. In one instance, the outlet end 224 can be removably coupled to a sample gas inlet 232 of a sample gas filter 106 to establish fluid communication (e.g., fluid flow path) between the sample line 108 and the sample gas filter 106.

Turning to the sample gas filter 106 of the sample gas filter assembly 104, the sample gas filter 106 can include a housing 230 that has a sample gas inlet 232 and a sample gas outlet 234. The sample gas inlet 232 can be in fluid communication with the sample gas outlet 234, thereby establishing a fluid flow path through the sample gas filter 106 (e.g., from the sample gas inlet 232 to the sample gas outlet 234).

The sample gas inlet 232 can receive sample gas into the sample gas filter 106. In one example, the sample gas inlet 232 can receive sample gas from a sample line 108 that is connected to an inspiratory line of a therapeutic gas delivery device (not shown in FIGS. 2A-2F). For example, the sample gas inlet 232 can be removably coupled to the sample line 108, which can be removably coupled to the inspiratory line, thereby establishing fluid communication between the inspiratory line and the sample gas filter 106.

The sample gas outlet 234 can discharge sample gas from the sample gas filter 106. In one example, the sample gas outlet 234 can discharge sample gas to a gas sensor module of a therapeutic gas delivery device (not shown in FIGS. 2A-2F). The sample gas outlet 234 can be configured to removably couple to a fitting (e.g., a luer fitting). For example, in one instance, the sample gas outlet 234 can extend outwards (e.g., away) from the sample gas filter 106 and can include external threads to removably couple to a fitting. In another instance (not shown in FIGS. 2A-2F), the sample gas outlet 234 can include internal threads to removably couple to a fitting. The configuration (e.g., external or internal threads) of the sample gas outlet 234 can be the opposite gender of the connector 228 of the sample line 108, so that the sample gas filter assembly 104 is unidirectional.

The housing 230 can define an exterior surface 236 and an interior surface 238 opposite the exterior surface 236, as illustrated, for example, in FIGS. 2E-2F. The housing 230 thickness can be defined by the distance between the exterior surface 236 and the interior surface 238. The housing 230 can have a substantially circular cross-section, which can define a diameter of the housing 230.

In one instance, the housing 230 can include a first shell 240 and a second shell 242, which can each define a portion of the exterior surface 236 and the interior surface 238 of the housing 230. The first shell 240 can define a first surface 244 opposite the sample gas inlet 232. The second shell 242 can define a second surface 246 opposite the sample gas outlet 234. The first surface 244 of the first shell 240 can, in whole or in part, abut the second surface 246 of the second shell 242 to form a housing 230 that is watertight. In one instance, an ultrasonic weld 248 can couple the first shell 240 and the second shell 242 together at the first surface 244 and the second surface 246.

A first chamber 250 can be located within the housing 230. In other words, the housing 230 of the sample gas filter 106 can define, in whole or in part, the first chamber 250. In one instance, the first chamber 250 is partially defined by the interior surface 238 of the first shell 240. In one instance, the first chamber 250 can have a substantially circular cross-section, which can define a diameter of the first chamber 250.

A second chamber 252 can be located within the housing 230. In other words, the housing 230 of the sample gas filter 106 can define, in whole or in part, the second chamber 252. In one instance, the second chamber 252 is partially defined by the interior surface 238 of the second shell 242. In one instance, the second chamber 252 can have a substantially circular cross-section, which can define a diameter of the second chamber 252.

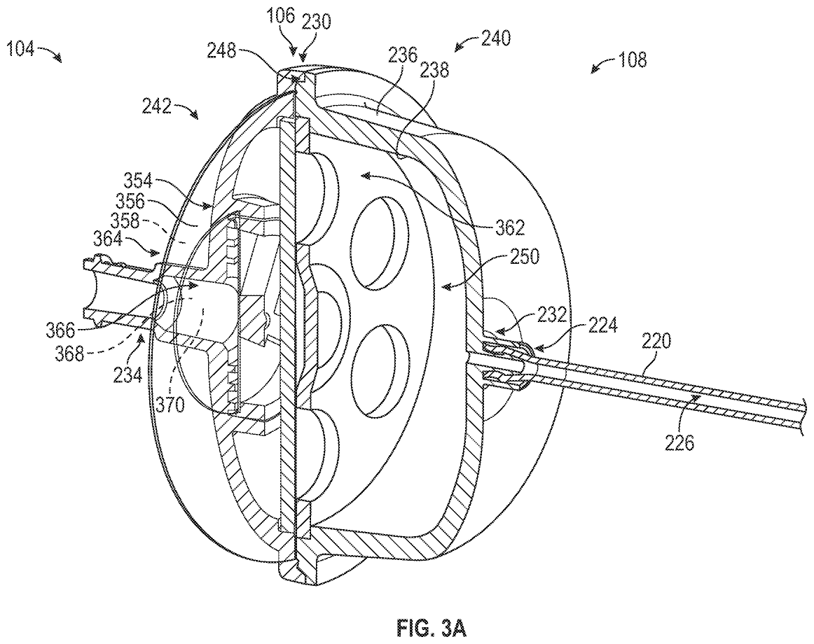

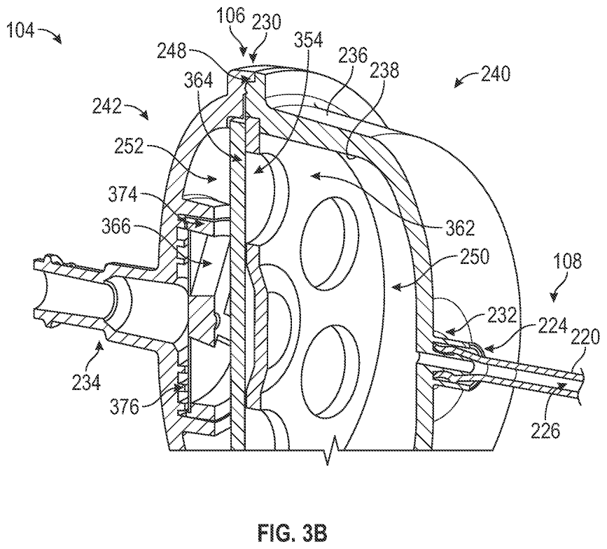

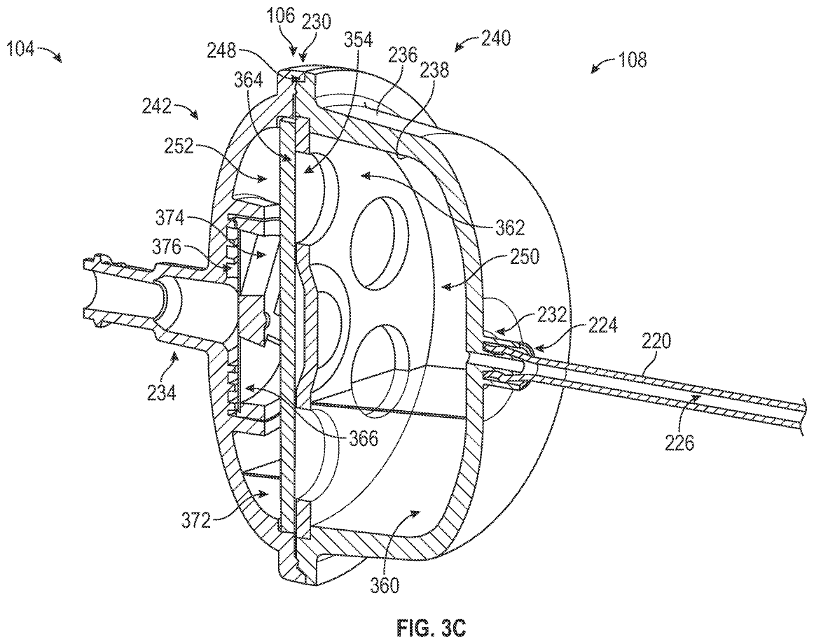

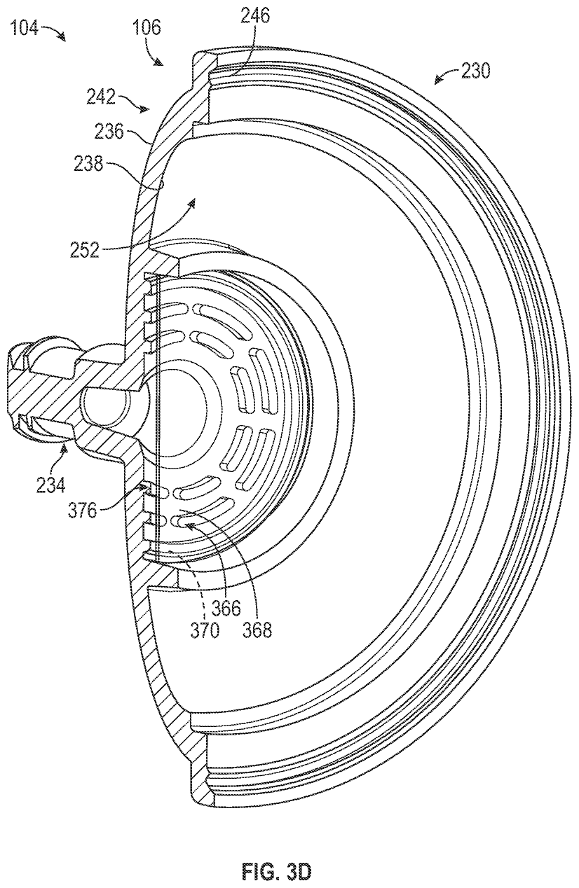

FIGS. 3A-3D illustrate three-dimensional, cross-sectional views of one instance of the sample gas filter 106 of a sample gas filter assembly 104. These figures illustrate various internal components, which can be included in the sample gas filter 106. For example, as illustrated in FIG. 3A, the sample gas filter 106 can include a first filter membrane 354 and a second filter membrane 366.

A first filter membrane 354 (e.g., first stage of filtration), as illustrated, for example, in the perspective view in FIG. 3A, can be included in the first chamber 250. The first filter membrane 354 can define a first side 356 and second side 358 opposite the first side 356. In one instance, the first filter membrane 354 can be substantially circular in shape, which can define a diameter of the first filter membrane 354. The first filter membrane 354 can remove vapor from the sample gas (e.g., sample gas flowing through the first filter membrane 354) and can cause liquid to coalesce and collect in a first reservoir 360 (e.g., front reservoir), as illustrated, for example, in FIG. 3C. In some instances, the first filter membrane 354 can be coalescing to liquid being both oleophobic and hydrophobic. In one instance, the first filter membrane 354 can be a glass fiber filter membrane.

In some instances, the first filter membrane 354 can be held in position (e.g., secured) and/or sealed by the abutment of the first surface 244 of the first shell 240 and the second surface 246 of the second shell 242. For example, an ultrasonic weld 248 that couples the first surface 244 and the second surface 246 can secure and/or seal the first filter membrane 354.

A first reservoir 360, as illustrated, for example, in FIG. 3C, can be included in the first chamber 250 (e.g., integrated into the housing 230). In some instances, the first reservoir 360 can be located between the sample gas inlet 232 and the first filter membrane 354. In other instances, the first reservoir 360 can be located between the sample gas inlet 232 and the baffle plate 362. The sample gas filter 106 can remove water vapor from the sample gas (e.g., via the first filter membrane 354) and collect the water in the first reservoir 360. The first reservoir 360 can be oriented axially along the length of the filter so that the sample gas filter 106 can operate (e.g., remove water vapor from the sample gas and collect the water) in any orientation during operation. The first reservoir 360 can define a volume and, in some instances, can be configured to accommodate water for approximately twelve hours of continuous use under humidification before it needs to be replaced.

A baffle plate 362 can, in some instances, be included in the first chamber 250 as illustrated, for example, in FIG. 3A. In some examples, the baffle plate 362 can mechanically support the first filter membrane 354 on the first side 356 (e.g., front side, which faces the flow of the sample gas) of the first filter membrane 354.

A fiber membrane 364 can, in some instances, be included in the first chamber 250. In some examples, the fiber membrane 364 can mechanically support the first filter membrane 354 on the second side 358 (e.g., backside) of the first filter membrane 354. In one instance, the fiber membrane 364 can be made of sintered porous plastic materials. In one example, the fiber membrane can be a course Vyon® fiber membrane.

A second filter membrane 366 (e.g., second stage of filtration), as illustrated, for example, in the perspective view in FIG. 3A, can be included in the second chamber 252. The second filter membrane 366 can define a first side 368 and second side 370 opposite the first side 368. In one instance, the second filter membrane 366 can be substantially circular in shape, which can define a diameter of the second filter membrane 366. The second filter membrane 366 can remove vapor from the sample gas (e.g., sample gas flowing through the second filter membrane 366) and cause liquid to coalesce and collect in a second reservoir 372 (e.g., rear reservoir), as illustrated, for example, in FIG. 3C. In some instances, the second filter membrane 366 can be a hydrophobic membrane. For example, the second filter membrane 366 can be a 0.22 μm Polytetrafluoroethylene (PTFE) filter membrane.

The diameter of the second filter membrane 366 can, in some examples, be smaller than the diameter of the first filter membrane 354. In other words, the diameter of the first filter membrane 354 can be larger than the diameter of the second filter membrane 366, as illustrated, for example, in FIG. 3A.

The first filter membrane 354 and the second filter membrane 366 can, in some instances, be separated to prevent wicking between the first filter membrane 354 and the second filter membrane 366. For example, a gap may exist between the first filter membrane 354 and the second filter membrane 366 to prevent wicking, as illustrated, for example, in FIG. 3C.

A second reservoir 372, as illustrated, for example, in FIG. 3C, can be included in the second chamber 252 (e.g., integrated into the housing 230). In some instances, the second reservoir 372 can be located between the first filter membrane 354 and the sample gas outlet 234. In other instances, the second reservoir 372 can be located between the fiber membrane 364 of the first chamber 250 and the press-fit baffle 374 (which is discussed below). The sample gas filter 106 can remove water vapor from the sample gas (e.g., via the second filter membrane 366) and collect the water in the second reservoir 372. The second reservoir 372 can be oriented axially along the length of the filter so that the sample gas filter 106 can operate (e.g., remove water vapor from the sample gas and collect the water) in any orientation during operation. The second reservoir 372 can define a volume and, in some instances, can be configured to accommodate water for approximately twelve hours of continuous use under humidification before it needs to be replaced.

The first reservoir 360 and the second reservoir 372 can be oriented axially so that the sample gas filter 106 can operate in any axial orientation. In some instances, the first reservoir 360 and the second reservoir 372 can be large enough (e.g., have enough volume) to accommodate water for at least twelve hours of continuous use. The volume of the second reservoir 372 can, in some instances, be smaller than the volume of the first reservoir 360. In other words, the volume of the first reservoir 360 can be larger than the volume of the second reservoir 372, as illustrated, for example, in FIG. 3C.

A press-fit baffle 374 can, in some instances, be included in the second chamber 252. In some examples, the press-fit baffle 374 can mechanically support the second filter membrane 366 on the first side 368 (e.g., front side, which faces the flow of the sample gas) of the second filter membrane 366.

A labyrinth support 376, as illustrated, for example, in FIG. 3D, can, in some instances, be included in the second chamber 252. The labyrinth support 376 can be located on the interior surface 238 of the housing 230 near the sample gas outlet 234 (e.g., the interior surface 238 of the second shell 242). In some examples, the labyrinth support 376 can mechanically support the second filter membrane 366 on the second side 370 (e.g., backside) of the second filter membrane 366. Additionally, the labyrinth support 376 can promote effective circulation of the sample gas behind the second filter membrane 366.

The sample gas filter assembly 104, including the sample line 108 and/or the sample gas filter 106, can be removed and replaced as needed. In other words, an existing sample line 108 and/or sample gas filter 106 can be removed from the therapeutic gas delivery device and a new sample line 108 and/or the sample gas filter 106 can be connected in its place, as previously described.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

Reference an “embodiment”, “aspect,” “instance,” or “example” means that a particular feature, structure, or characteristic described in connection with the embodiment is included in at least one embodiment of the disclosure. The appearances of the phrase in one “embodiment”, “aspect,” “instance,” or “example” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

What is claimed is:

1. A sample gas filter for a therapeutic gas delivery device, the sample gas filter comprising:
a housing having a sample gas inlet and a sample gas outlet, the sample gas inlet operable to receive a sample gas from a sample line connected to an inspiratory line of the therapeutic gas delivery device;
a first chamber disposed in the housing, the first chamber comprising:
a first filter membrane; and
a first reservoir disposed between the sample gas inlet and the first filter membrane; and
a second chamber disposed in the housing, the second chamber comprising:
a second filter membrane; and
a second reservoir disposed between the first filter membrane and the sample gas outlet,
wherein the first reservoir and the second reservoir are oriented axially such that the sample gas filter is operable to be used in any axial orientation, and
wherein the sample gas filter is operable to remove water vapor from the sample gas and collect water in the first reservoir and/or the second reservoir.

2. The sample gas filter of claim 1, wherein the first chamber further comprises:
a baffle plate operable to support the first filter membrane on a first side of the first filter membrane; and
a fiber membrane operable to support the first filter membrane on a second side of the first filter membrane.

3. The sample gas filter of claim 1, wherein the second chamber further comprises:
a press-fit baffle operable to support the second filter membrane on a first side of the second filter membrane; and
a labyrinth support on a wall of the housing having the sample gas outlet, the labyrinth support operable to support the second filter membrane on a second side of the second filter membrane.

4. The sample gas filter of claim 1, wherein the housing and each of the chambers have a substantially circular cross-section.

5. The sample gas filter of claim 1, wherein the first filter membrane and the second filter membrane are substantially circular and the first filter membrane has a larger diameter than the second filter membrane.

6. The sample gas filter of claim 1, wherein the first reservoir has a larger volume than the second reservoir.

7. The sample gas filter of claim 6, wherein the first reservoir and the second reservoir are large enough to accommodate water for 12 hours of continuous use.

8. The sample gas filter of claim 1, wherein the first filter membrane is a glass fiber filter membrane and the second filter membrane is a 0.22 μm PTFE filter membrane.

9. The sample gas filter of claim 2, wherein the fiber membrane is a course fiber membrane comprising sintered porous plastic.

10. The sample gas filter of claim 1, wherein the first filter membrane and the second filter membrane are separated to prevent wicking between the membranes.

11. A sample gas filter for a therapeutic gas delivery device, the sample gas filter comprising:
a housing having a sample gas inlet and a sample gas outlet, the sample gas inlet operable to receive a sample gas from a sample line connected to an inspiratory line of the therapeutic gas delivery device;
a first chamber disposed in the housing, the first chamber comprising:
a first filter membrane having a first side and a second side;
a baffle plate operable to support the first filter membrane on the first side;
a fiber membrane operable to support the first filter membrane on the second side; and
a first reservoir disposed between the sample gas inlet and the baffle plate; and
a second chamber disposed in the housing, the second chamber comprising:
a second filter membrane having a first side and a second side;
a press-fit baffle operable to support the second filter membrane on the first side;
a labyrinth support on a wall of the housing having the sample gas outlet, the labyrinth support operable to support the second filter membrane on the second side; and
a second reservoir disposed between the fiber membrane of the first chamber and the press-fit baffle.

12. The sample gas filter of claim 11, wherein the sample gas filter is operable to remove water vapor from the sample gas and collect water in the first reservoir and/or the second reservoir.

13. The sample gas filter of claim 11, wherein the housing and each of the chambers have a substantially circular cross-section.

14. The sample gas filter of claim 13, wherein the first reservoir and the second reservoir are oriented axially such that the sample gas filter is operable to be used in any axial orientation.

15. The sample gas filter of claim 11, wherein the first filter membrane and the second filter membrane are substantially circular and the first filter membrane has a larger diameter than the second filter membrane.

16. The sample gas filter of claim 11, wherein the first reservoir has a larger volume than the second reservoir.

17. The sample gas filter of claim 16, wherein the first reservoir and the second reservoir are large enough to accommodate water for 12 hours of continuous use.

18. The sample gas filter of claim 11, wherein the first filter membrane is a glass fiber filter membrane and the second filter membrane is a 0.22 μm PTFE filter membrane.

19. The sample gas filter of claim 11, wherein the fiber membrane is a course fiber membrane comprising sintered porous plastic materials.

20. The sample gas filter of claim 11, wherein the first filter membrane and the second filter membrane are separated to prevent wicking between the membranes.
